SEQUENCE LISTING (40 sequences)

SEQ ID NO: 1 - protein, 283 aa, synthetic construct
MSSLLLFNDKSRALQADIVAVQSQVVYGSVGNSIAVPAIKQNGLNVFAVPTVLLSNTPHYDTFYGGAIPDEWFSGYLRALQERDALRQLRAVTTGYMGTASQIKILAEWLTALRKDHPDLLIMVDPVIGDIDSGIYVKPDLPEAYRQYLLPLAQGITPNIFELEILTGKNCRDLDSAIAAAKSLLSDTLKWVVVTSASGNEENQEMQVVVVTADSVNVISHSRVKTDLKGTGDLFCAQLISGLLKGKALTDAVHRAGLRVLEVMRYTQQHESDELILPPLAEA

SEQ ID NO: 2 - DNA, 849 bp, synthetic construct
  source 1..849: mol_type = other DNA
atgtcaagtctattattgtttaatgataaatctcgtgctcttcaggcagatattgttgcagtgcagagccaagttgtgtacggctctgtcggtaatagcattgcggtgccggcaattaaacagaacggtctgaacgtgttcgcggtaccgaccgttctgctttcgaacacgccgcactacgataccttctatggcggtgctatcccggacgaatggtttagcggttacctgcgggcgctgcaagagcgcgatgcgttgagacaactgcgcgccgtgaccaccggctacatgggtactgcgagccagattaaaatcctggctgagtggctgacggccctgcgcaaggaccacccggacctgttgatcatggttgatccggttatcggcgacatcgactccggcatctacgtgaagccggacctgccggaagcatatcgtcaatatttgctgccactcgcccaaggcattaccccgaatatttttgagctggaaatcctgaccggcaaaaactgtcgtgacctggattccgcgatcgcggcggcaaaaagcctgctgtccgatacgctgaagtgggttgtcgtcacttcagctagcggtaacgaggaaaaccaggagatgcaggttgtcgttgtgaccgcggactctgttaatgtgatctcccacagccgtgtgaaaaccgatctgaagggtacaggtgacttgttctgcgcacagctcatcagcggcttgctgaagggtaaggcgctgaccgacgcggttcatcgtgccggcctgcgtgtattggaggtgatgcgttatacccagcagcatgaaagcgatgagctgattttgccgcctctcgcggaagct

SEQ ID NO: 3 - DNA, 29 bp, synthetic construct
  source 1..29: mol_type = other DNA
cgagacagaacttaatgggcccgctaaca

SEQ ID NO: 4 - DNA, 29 bp, synthetic construct
  source 1..29: mol_type = other DNA
tgttagcgggcccattaagttctgtctcg

SEQ ID NO: 5 - DNA, 33 bp, synthetic construct
  source 1..33: mol_type = other DNA
gtgaaaaccgatctgatgggtacaggtgacttg

SEQ ID NO: 6 - DNA, 33 bp, synthetic construct
  source 1..33: mol_type = other DNA
caagtcacctgtacccatcagatcggttttcac

SEQ ID NO: 7 - DNA, 33 bp, synthetic construct
  source 1..33: mol_type = other DNA
gtgaaaaccgatctggaaggtacaggtgacttg

SEQ ID NO: 8 - DNA, 33 bp, synthetic construct
  source 1..33: mol_type = other DNA
caagtcacctgtaccttccagatcggttttcac

SEQ ID NO: 9 - DNA, 33 bp, synthetic construct
  source 1..33: mol_type = other DNA
gtgaaaaccgatctggatggtacaggtgacttg

SEQ ID NO: 10 - DNA, 33 bp, synthetic construct
  source 1..33: mol_type = other DNA
caagtcacctgtaccatccagatcggttttcac

SEQ ID NO: 11 - DNA, 33 bp, synthetic construct
  source 1..33: mol_type = other DNA
gtgaaaaccgatctggcaggtacaggtgacttg

SEQ ID NO: 12 - DNA, 33 bp, synthetic construct
  source 1..33: mol_type = other DNA
caagtcacctgtacctgccagatcggttttcac

SEQ ID NO: 13 - DNA, 33 bp, synthetic construct
  source 1..33: mol_type = other DNA
gtgaaaaccgatctggtaggtacaggtgacttg

SEQ ID NO: 14 - DNA, 33 bp, synthetic construct
  source 1..33: mol_type = other DNA
caagtcacctgtacctaccagatcggttttcac

SEQ ID NO: 15 - DNA, 33 bp, synthetic construct
  source 1..33: mol_type = other DNA
gtgaaaaccgatctgaatggtacaggtgacttg

SEQ ID NO: 16 - DNA, 33 bp, synthetic construct
  source 1..33: mol_type = other DNA
caagtcacctgtaccattcagatcggttttcac

SEQ ID NO: 17 - DNA, 33 bp, synthetic construct
  source 1..33: mol_type = other DNA
gtgaaaaccgatctgactggtacaggtgacttg

SEQ ID NO: 18 - DNA, 33 bp, synthetic construct
  source 1..33: mol_type = other DNA
caagtcacctgtaccagtcagatcggttttcac

SEQ ID NO: 19 - DNA, 33 bp, synthetic construct
  source 1..33: mol_type = other DNA
gtgaaaaccgatctgattggtacaggtgacttg

SEQ ID NO: 20 - DNA, 33 bp, synthetic construct
  source 1..33: mol_type = other DNA
caagtcacctgtaccaatcagatcggttttcac

SEQ ID NO: 21 - DNA, 33 bp, synthetic construct
  source 1..33: mol_type = other DNA
gtgaaaaccgatctgcgtggtacaggtgacttg

SEQ ID NO: 22 - DNA, 33 bp, synthetic construct
  source 1..33: mol_type = other DNA
caagtcacctgtaccacgcagatcggttttcac

SEQ ID NO: 23 - DNA, 33 bp, synthetic construct
  source 1..33: mol_type = other DNA
gtgaaaaccgatctgcatggtacaggtgacttg

SEQ ID NO: 24 - DNA, 33 bp, synthetic construct
  source 1..33: mol_type = other DNA
caagtcacctgtaccatgcagatcggttttcac

SEQ ID NO: 25 - DNA, 33 bp, synthetic construct
  source 1..33: mol_type = other DNA
gtgaaaaccgatctgcctggtacaggtgacttg

SEQ ID NO: 26 - DNA, 33 bp, synthetic construct
  source 1..33: mol_type = other DNA
caagtcacctgtaccaggcagatcggttttcac

SEQ ID NO: 27 - DNA, 33 bp, synthetic construct
  source 1..33: mol_type = other DNA
gtgaaaaccgatctgtggggtacaggtgacttg

SEQ ID NO: 28 - DNA, 33 bp, synthetic construct
  source 1..33: mol_type = other DNA
caagtcacctgtaccccacagatcggttttcac

SEQ ID NO: 29 - DNA, 33 bp, synthetic construct
  source 1..33: mol_type = other DNA
gtgaaaaccgatctgtgtggtacaggtgacttg

SEQ ID NO: 30 - DNA, 33 bp, synthetic construct
  source 1..33: mol_type = other DNA
caagtcacctgtaccacacagatcggttttcac

SEQ ID NO: 31 - DNA, 33 bp, synthetic construct
  source 1..33: mol_type = other DNA
gtgaaaaccgatctgtatggtacaggtgacttg

SEQ ID NO: 32 - DNA, 33 bp, synthetic construct
  source 1..33: mol_type = other DNA
caagtcacctgtaccatacagatcggttttcac

SEQ ID NO: 33 - DNA, 33 bp, synthetic construct
  source 1..33: mol_type = other DNA
gtgaaaaccgatctgtctggtacaggtgacttg

SEQ ID NO: 34 - DNA, 33 bp, synthetic construct
  source 1..33: mol_type = other DNA
caagtcacctgtaccagacagatcggttttcac

SEQ ID NO: 35 - DNA, 33 bp, synthetic construct
  source 1..33: mol_type = other DNA
gtgaaaaccgatctgtttggtacaggtgacttg

SEQ ID NO: 36 - DNA, 33 bp, synthetic construct
  source 1..33: mol_type = other DNA
caagtcacctgtaccaaacagatcggttttcac

SEQ ID NO: 37 - DNA, 33 bp, synthetic construct
  source 1..33: mol_type = other DNA
gtgaaaaccgatctgggcggtacaggtgacttg

SEQ ID NO: 38 - DNA, 33 bp, synthetic construct
  source 1..33: mol_type = other DNA
caagtcacctgtaccgcccagatcggttttcac

SEQ ID NO: 39 - DNA, 33 bp, synthetic construct
  source 1..33: mol_type = other DNA
gtgaaaaccgatctgcttggtacaggtgacttg

SEQ ID NO: 40 - DNA, 33 bp, synthetic construct
  source 1..33: mol_type = other DNA
caagtcacctgtaccaagcagatcggttttcac

(19) United States
(12) Patent Application Publication
(10) Pub. No.: US 2026/0185134 A1
(43) Pub. Date: Jul. 2, 2026

(54) PYRIDOXAL KINASE MUTANT

(71) Applicants: TAIZHOU UNIVERSITY, Taizhou (CN); Taizhou Lingfeng Biotechnology Co., Ltd., Taizhou (CN)

(72) Inventors: Zhongyi YANG, Taizhou (CN); Fei WANG, Taizhou (CN); Qingfeng CAI, Taizhou (CN); Yi WEI, Taizhou (CN); Weijin HUANG, Taizhou (CN)

(73) Assignees: TAIZHOU UNIVERSITY, Taizhou (CN); Taizhou Lingfeng Biotechnology Co., Ltd., Taizhou (CN)

(21) Appl. No.: 19/435,805

(22) Filed: Dec. 30, 2025

(30) Foreign Application Priority Data
Dec. 31, 2024 (CN) 202411985002.0

(51) Int. Cl. C12P 17/12; C12N 9/12

(52) U.S. Cl. CPC C12P 17/12; C12N 9/1223

(57) ABSTRACT

A pyridoxal kinase mutant is provided. To solve the problem of low concentration of existing enzyme catalyzed products, provided is a pyridoxal kinase mutant having an amino acid sequence selected from an amino acid sequence as shown in SEQ ID NO: 1 in which lysine at position 229 is mutated to alanine, phenylalanine, arginine, threonine, histidine, serine, tyrosine, valine, leucine, isoleucine, proline, aspartic acid, or glutamic acid; and the pyridoxal kinase mutant is used to catalyze the synthesis of pyridoxal phosphate from pyridoxal.

### CROSS REFERENCE TO THE RELATED APPLICATIONS

This application is based upon and claims priority to Chinese Patent Application No. 202411985002.0, filed on Dec. 31, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named YFZT0202S_Sequence_Listing.xml, created on 12/22/2025, and is 37,620 bytes in size.

### TECHNICAL FIELD

The present disclosure relates to a pyridoxal kinase mutant, a recombinant expression vector and a microbial cell and application of the pyridoxal kinase mutant, the recombinant expression vector and the microbial cell, and belongs to the technical field of biocatalysis.

### BACKGROUND

Pyridoxal phosphate is a very important biochemical substance in the metabolic process of organisms, is an active form of vitamin B6, and is also a coenzyme for many enzymatic reactions, especially those involving amino. Pyridoxal phosphate also has a variety of uses in medicine, and may be used in the treatment of diseases such as anemia and atherosclerosis, and prevention of cardiovascular and cerebrovascular diseases and the like. Therefore, pyridoxal phosphate has wide application prospects.

Currently, pyridoxal phosphate (PLP) can be synthesized by phosphorylating pyridoxal by a chemical process or an enzymatic process. The chemical process of pyridoxal phosphate requires the use of far excess phosphorus pentoxide and phosphoric acid, producing a large amount of highly concentrated phosphorus-containing waste water, resulting in high production costs. Whereas the enzymatic process mainly uses ATP as a phosphate donor and is catalyzed by pyridoxal kinase, a reaction can be carried out at normal temperature, the process has high potential advantages, but the concentration of a main product pyridoxal phosphate of the reaction is low, which is a main reason affecting the industrial application of enzymatic synthesis of PLP.

The technical level of the enzymatic synthesis of pyridoxal phosphate in the existing literature is low. For example, it is reported in the Chinese patent application (publication No.: CN107236770A) that about 100 g/L of pyridoxal hydrochloride can be subjected to an enzymatic reaction to obtain pyridoxal phosphate, but a specific enzyme raw material adopted for pyridoxal is not disclosed in the literature and there is no reference. For another example, in a mutant of pyridoxal kinase and application thereof disclosed in Chinese patent document (publication No.: CN117467641A), the mutant of pyridoxal kinase mentioned is also produced by site-directed mutation at least at position 135 based on a wild-type amino acid sequence, and also disclosed is the mutant of pyridoxal kinase further including a mutation of an amino acid at least one of the following positions: position 29, position 52, position 57, position 195, position 209, position 210, position 220, position 237, position 261, and position 277, but more importantly, although this mutant is mentioned in this document, it can be seen from the full text that although the document mentions that the pyridoxal kinase has enzyme activity in pyridoxal catalysis, it cannot be concluded that a conversion rate for the catalytic synthesis of pyridoxal phosphate by using the pyridoxal kinase is high, not necessarily having a high product yield. Instead, further research is needed for confirmation. In fact, the document discloses that pyridoxine phosphate is produced from pyridoxine by enzymatic catalysis using the pyridoxal kinase, and pyridoxine phosphate is further oxidized into pyridoxal phosphate. In essence, pyridoxal phosphate is synthesized by a combination of the enzymatic process and the chemical process, the ability of the pyridoxal kinase to convert pyridoxal to pyridoxal phosphate and the product yield are not disclosed, and it is also disclosed that the effect on enzyme activity is better by simultaneous mutations at multiple amino acid sites, all also for the synthesis of pyridoxine phosphate.

### SUMMARY

In view of the defects existing in the prior art, the present disclosure provides a pyridoxal kinase mutant, a recombinant expression vector and a microbial cell and application of the pyridoxal kinase mutant, the recombinant expression vector and the microbial cell, and solves the problem of a low product conversion rate of catalyzing pyridoxal phosphorylation through the existing pyridoxal kinase by providing a new mutant enzyme.

One objective of the present disclosure is achieved by the following technical solution: provided is a pyridoxal kinase mutant, having an amino acid sequence selected from an amino acid sequence as shown in SEQ ID NO: 1 in which lysine at position 229 is mutated to alanine, phenylalanine, methionine, arginine, threonine, histidine, serine, tyrosine, valine, leucine, isoleucine, proline, asparagine, aspartic acid, or glutamic acid. The pyridoxal kinase mutant is used to catalyze the synthesis of pyridoxal phosphate from pyridoxal.

In the actual research and development process of the present disclosure, it is found that although the catalytic efficiency may be reduced after an amino acid at position 229 of wild-type pyridoxal kinase is mutated, the pyridoxal kinase mutant can greatly improve the product concentration for the catalytic synthesis of pyridoxal phosphate from pyridoxal, with the quality effect of a high product concentration and a high product yield. In particular, in the present disclosure, by selective mutation at position 229 in an amino acid sequence of the wild-type pyridoxal kinase to the amino acid described above, the obtained mutants all have better enzyme activity. More importantly, the mutant not only has good enzyme activity, but also can directly convert pyridoxal to pyridoxal phosphate by enzyme-catalyzed phosphorylation, with a high product conversion rate, and the concentration of the obtained pyridoxal phosphate is high, and is obviously improved compared with that of a product obtained by catalyzing pyridoxal phosphorylation through the wild-type pyridoxal kinase. Meanwhile, the mutant of the present disclosure can efficiently convert pyridoxal into pyridoxal phosphate directly by enzymatic catalysis only by mutating at position 229 of the above amino acid sequence as shown in SEQ ID NO: 1, without mutating at other positions, and has a high selectivity effect.

The description of mutating the lysine (K) at position 229 to other amino acids can be expressed in the following manners:
when the lysine at position 229 is mutated to alanine, the mutant can be described as K229A;
when the lysine at position 229 is mutated to phenylalanine, the mutant can be described as K229F;
when the lysine at position 229 is mutated to methionine, the mutant can be described as K229M;
when the lysine at position 229 is mutated to arginine, the mutant can be described as K229R;
when the lysine at position 229 is mutated to threonine, the mutant can be described as K229T;
when the lysine at position 229 is mutated to histidine, the mutant can be described as K229H;
when the lysine at position 229 is mutated to serine, the mutant can be described as K229S;
when the lysine at position 229 is mutated to tyrosine, the mutant can be described as K229Y;
when the lysine at position 229 is mutated to valine, the mutant can be described as K229V;
when the lysine at position 229 is mutated to leucine, the mutant can be described as K229L;
when the lysine at position 229 is mutated to isoleucine, the mutant can be described as K229I;
when the lysine at position 229 is mutated to proline, the mutant can be described as K229P;
when the lysine at position 229 is mutated to asparagine, the mutant can be described as K229N;
when the lysine at position 229 is mutated to aspartic acid, the mutant can be described as K229D; and
when the lysine at position 229 is mutated to glutamic acid, the mutant can be described as K229E.

The concentration of a product obtained by catalyzing pyridoxal phosphorylation through the preferred pyridoxal kinase mutant described above can be further increased compared with the wild-type pyridoxal kinase, resulting in an increase in the concentration of the product pyridoxal phosphate to a level of 2 times or more.

In order to better improve the conversion ability of the selected pyridoxal kinase mutant to catalyze pyridoxal phosphorylation and to have a high product concentration level, further preferably, the amino acid sequence of the pyridoxal kinase mutant is the amino acid sequence as shown in SEQ ID NO: 1 in which lysine at position 229 is mutated to alanine, phenylalanine, histidine, serine or tyrosine.

That is, correspondingly, when the lysine at position 229 is mutated to alanine, the mutant can be described as K229A;
when the lysine at position 229 is mutated to phenylalanine, the mutant can be described as K229F;
when the lysine at position 229 is mutated to histidine, the mutant can be described as K229H;
when the lysine at position 229 is mutated to serine, the mutant can be described as K229S; and
when the lysine at position 229 is mutated to tyrosine, the mutant can be described as K229Y.

In the pyridoxal kinase mutant described above, the pyridoxal kinase mutant is produced by mutating lysine at position 229 of an amino acid sequence derived from Escherichia coli having an amino acid sequence as shown in SEQ ID NO: 1 and a nucleotide sequence as shown in SEQ ID NO: 2. Using Escherichia coli as a basis for mutation is equivalent to mutation on the basis of the wild-type pyridoxal kinase, with better mutability, and mutation is performed on the basis of this amino acid sequence, and finally only one site at position 229 needs to be mutated, so that the resulting mutant has a high enzyme catalytic capacity for pyridoxal and has the effect of high pyridoxal phosphate product concentration. It can also be more directly explained that a product conversion rate of phosphorylating pyridoxal by the mutant of the present disclosure is far superior to the catalytic conversion capacity of the wild-type enzyme, achieving better application values.

A further objective of the present disclosure is achieved by the following technical solution: provided is a recombinant expression vector carrying the pyridoxal kinase mutant described above. Since the above-mentioned pyridoxal kinase mutant of the present disclosure has excellent enzyme activity and ability to catalyze the direct conversion of pyridoxal to pyridoxal phosphate, and has the advantage of a high pyridoxal phosphate product yield, the recombinant expression vector carrying the above-mentioned pyridoxal kinase mutant can also achieve the above-mentioned effects. Selection of the recombinant expression vector can be accomplished by using a variety of expression vectors including, but not limited to, the following PET expression vectors, PUC expression vectors, and the like. However, in order to better realize the above expression vector, further preferably, the recombinant expression vector uses PET-30a as an expression vector.

A further objective of the present disclosure is achieved by the following technical solution: provided is a microbial cell having a microorganism as an expression host, the microorganism carrying the pyridoxal kinase mutant or carrying the recombinant expression vector containing the pyridoxal kinase mutant. The pyridoxal kinase mutant has an amino acid sequence selected from an amino acid sequence as shown in SEQ ID NO: 1 in which lysine at position 229 is mutated to alanine, phenylalanine, methionine, arginine, threonine, histidine, serine, tyrosine, valine, leucine, isoleucine, proline, asparagine, aspartic acid, or glutamic acid; and the pyridoxal kinase mutant is used to catalyze the synthesis of pyridoxal phosphate from pyridoxal.

Similarly, since the pyridoxal kinase mutant of the present disclosure has excellent enzyme activity and ability to catalyze the direct conversion of pyridoxal to pyridoxal phosphate, and has the advantage of high pyridoxal phosphate product concentration, the microbial cell carrying the pyridoxal kinase mutant or the recombinant expression vector can also achieve the above effects, and has the effect of efficiently catalyzing the synthesis of pyridoxal phosphate from pyridoxal. For the microorganism as the expression host, genetically engineered bacteria such as Escherichia coli and Bacillus subtilis can be used. Further preferably, the microorganism is Escherichia coli.

In the above microbial cell, preferably, the pyridoxal kinase mutant is produced by mutating lysine at position 229 of an amino acid sequence derived from Escherichia coli having an amino acid sequence as shown in SEQ ID NO: 1 and a nucleotide sequence as shown in SEQ ID NO: 2.

A further objective of the present disclosure is achieved by the following technical solution: provided is application of the pyridoxal kinase mutant, the recombinant expression vector and the microbial cell, wherein pyridoxal phosphate is prepared by phosphorylating a substrate pyridoxal or an acid salt of pyridoxal under the action of pyridoxal kinase selected from one or more of the pyridoxal kinase mutant, the recombinant expression vector and the microbial cell. Since the pyridoxal kinase mutant of the present disclosure can efficiently convert pyridoxal to pyridoxal phosphate by enzymatic catalysis, the expression vector or microorganism carrying the above pyridoxal kinase mutant can also achieve the performance of phosphorylating pyridoxal efficiently, and also has the effect of high product concentration, and the concentration of pyridoxal phosphate can be increased to 2 times or more compared with the enzyme catalytic conversion capacity of the wild-type pyridoxal kinase.

In the application of the pyridoxal kinase mutant, the recombinant expression vector and the microbial cell, preferably, pyridoxal phosphate is specifically prepared by:
carrying out a reaction in a reaction system of pyridoxal hydrochloride, a magnesium salt, ATP, pyridoxal kinase, acetate kinase and ACP. The above-mentioned raw materials such as acetate kinase, ATP (an adenosine triphosphate disodium salt), and ACP (an acetyl phosphate diammonium salt) are commercially available directly, pyridoxal kinase, i.e., the pyridoxal kinase mutant, the recombinant expression vector and the microbial cell described above realize the regeneration of ADP to ATP by introducing an ATP regeneration system and using relatively cheap acetyl phosphate, so that the reaction is carried out more efficiently and the yield of the product can also be better guaranteed, the concentration of the obtained pyridoxal phosphate is high, which is more conducive to industrial production. Furthermore, in the present disclosure, although some mutants may reduce the catalytic speed after the amino acid at position 229 of the wild-type pyridoxal kinase is mutated, the inhibitory effect of high-concentration substrates and products on the enzyme catalytic activity can be avoided; in combination with the use of the ATP regenerating system to change the equilibrium state of the reaction from the reaction kinetics, the above-described pyridoxal kinase mutant can better break through the self-inhibition of the enzymatic reaction by pyridoxal and pyridoxal phosphate, ultimately catalyzing the synthesis to obtain a pyridoxal phosphate product with high concentration. Preferably, the magnesium salt is selected from magnesium sulfate or magnesium chloride and the like, and magnesium sulfate may be selected from magnesium sulfate heptahydrate and the like. Preferably, the reaction is carried out at a pH of 5-6, and the pH of the system may be adjusted by using an alkali metal hydroxide, such as an aqueous hydroxide solution or the like.

In the application of the pyridoxal kinase mutant, the recombinant expression vector and the microbial cell described above, preferably, a molar ratio of pyridoxal to the ATP to the ACP to the magnesium salt is 1:(0.05-0.5):(1.0-6.0):(0.01-0.20).

The amount of the pyridoxal kinase and the acetate kinase used here may be in a catalytic amount, for example, in order to ensure that the reaction is carried out under conditions with enzyme activity, preferably, a weight ratio of pyridoxal to an enzyme solution of the pyridoxal kinase to an enzyme solution of the acetate kinase is 1:(0.2-5.0):(0.1-5.0).

In the application of the pyridoxal kinase mutant, the recombinant expression vector and the microbial cell as described above, preferably, the reaction is carried out at a temperature controlled to be 30-37° C.

A reaction equation for pyridoxal phosphate can be expressed as follows:

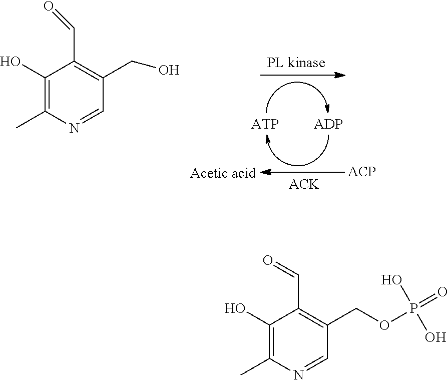 [SMILES: Cc1ncc(CO)c(C=O)c1O ; Cc1ncc(COP(=O)(O)O)c(C=O)c1O]

In summary, compared with the prior art, the present disclosure has the following advantages:

1. The pyridoxal kinase mutant of the present disclosure can directly convert pyridoxal to pyridoxal phosphate by enzyme-catalyzed phosphorylation, and has the effect of a high product conversion rate, and the concentration of the product is obviously improved compared with that of a product obtained by catalyzing pyridoxal phosphorylation through the wild-type pyridoxal kinase.

2. The mutant of the present disclosure can efficiently convert pyridoxal into pyridoxal phosphate directly by enzymatic catalysis only by mutating at position 229 of the amino acid sequence as shown in SEQ ID NO: 1, without mutating at other positions, and has a high selectivity effect, and the concentration of the obtained pyridoxal phosphate is high.

### BRIEF DESCRIPTION OF THE DRAWINGS

FIG. 1 is an SDS-PAGE image of protein expression of wild-type PdxK and a pyridoxal kinase mutant K229F.

FIG. 2 is a curve graph of a reaction process for the catalytic synthesis of PLP by wild-type PdxK.

FIG. 3 is a curve graph of a reaction process for the catalytic synthesis of PLP by the pyridoxal kinase mutant K229F.

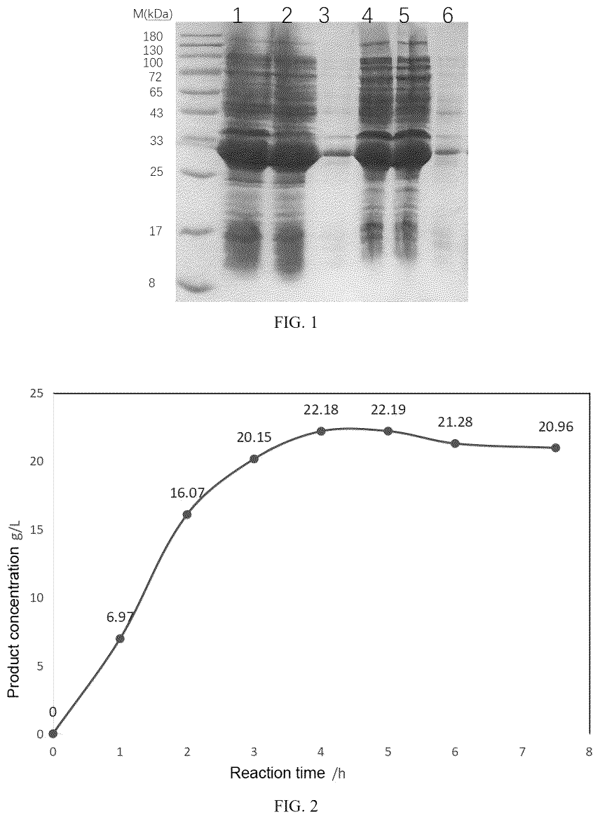

In FIG. 1, 1. disrupted total proteins after induction of wild-type PdxK; 2. a supernatant obtained after induction and disruption of wild-type PdxK; 3. a pellet obtained after induction and disruption of wild-type PdxK; 4. total proteins after induction of a pyridoxal kinase mutant K229F; 5. a supernatant obtained after induction and disruption of the pyridoxal kinase mutant K229F; and 6. a pellet obtained after induction and disruption of the mutant K229F.

### DETAILED DESCRIPTION OF THE EMBODIMENTS

The technical solutions of the present disclosure are further specifically illustrated below by specific examples and the drawings, but the present disclosure is not limited to these examples.

### Example 1

### Fermentation of Pyridoxal Kinase (PdxK) Wild-Type Engineered Bacteria in a 1L Shake Flask, and Enzyme Activity Assay

A wild-type PdxK glycerol stock was inoculated into 5 Ml of an LB liquid medium containing kanamycin at a final concentration of 50 g/Ml at an inoculation amount of 0.1% v/v, and was cultured with shaking at 200 rpm at 37° C. for 12 h, 5 Ml of a liquid culture was inoculated into 200 Ml of a TB liquid medium containing kanamycin at a final concentration of 50 g/Ml at an inoculation amount of 1% v/v, and was cultured with shaking at 200 rpm at 37° C. for 4-6 h until an OD value reached about 0.8, induction was performed by adding 1 Mm IPTG at 25° C. for 12 h, centrifugation was performed at 8000 rpm at 4° C. for 10 min, a supernatant was discarded, and wet bacteria were collected. The collected wet bacteria were further wall-broken by an ultrasonic cell disruptor to obtain a cell lysate, i.e., a crude enzyme solution of PdxK.

The wild-type PdxK is derived from Escherichia coli having an amino acid sequence as shown in SEQ ID NO: 1 and a nucleotide sequence as shown in SEQ ID NO: 2.

### Example 2

### Cloning Process of the Pyridoxal Kinase Mutant

Primers were designed, and a gene template Pet-30a (+) for an expression vector Pet30 (a)+was downloaded from the SnapGene official website. A PdxK sequence was inserted into the vector through two restriction sites “Hindlll” and “Ndel”. An amino acid at position 229 (K229) in a sequence of interest was mutated, and a primer sequence having a length of 30-36 bp and a Tm value within 55-65° C. was selected. The designed primers were synthesized according to conventional methods, and the obtained mutation primers are shown in Table 1:

TABLE 1

| | | |
|---|---|---|
| F-2 | CGAGACAGAACTTAATGGGCCCGCTAACA | SEQ ID NO: 3 |
| R-2 | TGTTAGCGGGCCCATTAAGTTCTGTCTCG | SEQ ID NO: 4 |
| M-F1 | GTGAAAACCGATCTGATGGGTACAGGTGACTTG | SEQ ID NO: 5 |
| M-R1 | CAAGTCACCTGTACCCATCAGATCGGTTTTCAC | SEQ ID NO: 6 |
| E-F1 | GTGAAAACCGATCTGGAAGGTACAGGTGACTTG | SEQ ID NO: 7 |
| E-R1 | CAAGTCACCTGTACCTTCCAGATCGGTTTTCAC | SEQ ID NO: 8 |
| D-F1 | GTGAAAACCGATCTGGATGGTACAGGTGACTTG | SEQ ID NO: 9 |
| D-R1 | CAAGTCACCTGTACCATCCAGATCGGTTTTCAC | SEQ ID NO: 10 |
| A-F1 | GTGAAAACCGATCTGGCAGGTACAGGTGACTTG | SEQ ID NO: 11 |
| A-R1 | CAAGTCACCTGTACCTGCCAGATCGGTTTTCAC | SEQ ID NO: 12 |
| V-F1 | GTGAAAACCGATCTGGTAGGTACAGGTGACTTG | SEQ ID NO: 13 |
| V-R1 | CAAGTCACCTGTACCTACCAGATCGGTTTTCAC | SEQ ID NO: 14 |
| N-F1 | GTGAAAACCGATCTGAATGGTACAGGTGACTTG | SEQ ID NO: 15 |
| N-R1 | CAAGTCACCTGTACCATTCAGATCGGTTTTCAC | SEQ ID NO: 16 |
| T-F1 | GTGAAAACCGATCTGACTGGTACAGGTGACTTG | SEQ ID NO: 17 |
| T-R1 | CAAGTCACCTGTACCAGTCAGATCGGTTTTCAC | SEQ ID NO: 18 |
| I-F1 | GTGAAAACCGATCTGATTGGTACAGGTGACTTG | SEQ ID NO: 19 |
| I-R1 | CAAGTCACCTGTACCAATCAGATCGGTTTTCAC | SEQ ID NO: 20 |
| R-F1 | GTGAAAACCGATCTGCGTGGTACAGGTGACTTG | SEQ ID NO: 21 |
| R-R1 | CAAGTCACCTGTACCACGCAGATCGGTTTTCAC | SEQ ID NO: 22 |
| H-F1 | GTGAAAACCGATCTGCATGGTACAGGTGACTTG | SEQ ID NO: 23 |
| H-R1 | CAAGTCACCTGTACCATGCAGATCGGTTTTCAC | SEQ ID NO: 24 |
| P-F1 | GTGAAAACCGATCTGCCTGGTACAGGTGACTTG | SEQ ID NO: 25 |
| P-R1 | CAAGTCACCTGTACCAGGCAGATCGGTTTTCAC | SEQ ID NO: 26 |
| W-F1 | GTGAAAACCGATCTGTGGGGTACAGGTGACTTG | SEQ ID NO: 27 |
| W-R1 | CAAGTCACCTGTACCCCACAGATCGGTTTTCAC | SEQ ID NO: 28 |
| C-F1 | GTGAAAACCGATCTGTGTGGTACAGGTGACTTG | SEQ ID NO: 29 |
| C-R1 | CAAGTCACCTGTACCACACAGATCGGTTTTCAC | SEQ ID NO: 30 |
| Y-F1 | GTGAAAACCGATCTGTATGGTACAGGTGACTTG | SEQ ID NO: 31 |
| Y-R1 | CAAGTCACCTGTACCATACAGATCGGTTTTCAC | SEQ ID NO: 32 |
| S-F1 | GTGAAAACCGATCTGTCTGGTACAGGTGACTTG | SEQ ID NO: 33 |
| S-R1 | CAAGTCACCTGTACCAGACAGATCGGTTTTCAC | SEQ ID NO: 34 |
| F-F1 | GTGAAAACCGATCTGTTTGGTACAGGTGACTTG | SEQ ID NO: 35 |
| F-R1 | CAAGTCACCTGTACCAAACAGATCGGTTTTCAC | SEQ ID NO: 36 |
| G-F1 | GTGAAAACCGATCTGGGCGGTACAGGTGACTTG | SEQ ID NO: 37 |
| G-R1 | CAAGTCACCTGTACCGCCCAGATCGGTTTTCAC | SEQ ID NO: 38 |
| L-F1 | GTGAAAACCGATCTGCTTGGTACAGGTGACTTG | SEQ ID NO: 39 |
| L-R1 | CAAGTCACCTGTACCAAGCAGATCGGTTTTCAC | SEQ ID NO: 40 |

Amplification conditions: initial denaturation at 95° C. for 3 min, followed by denaturation at 95° C. for 30 sec, annealing at 59° C. for 30 sec, and extension at 72° C. for 45 s (a short fragment)/3 min (a long fragment) for a total of 30 cycles, and finally amplification at 72° C. for 10 min.

After the reaction was completed, PCR amplification products were detected by 1% agarose gel electrophoresis, and target bands meeting the expected results were obtained. According to the standard operation of a kit, this target fragment was recovered and purified, the target gene fragment was then ligated to the vector by using an Exnase II ligase to obtain a ligation product, i.e., a recombinant expression vector, the resulting ligation product was transformed into competent cells of E. coli BL21 (DE3), the transformed cells were plated on an LB solid plate containing 50 g/Ml kanamycin, and a plurality of well-grown single colonies were picked, inoculated into an LB liquid medium containing kanamycin at a final concentration of 50 g/Ml, incubated at 37° C. at 200 rpm for 12-16 h, and prepared into a glycerol stock to be stored at −80° C. for later use. A plasmid extracted from the remaining culture was sent for sequencing. The results showed that a cloned K229 gene sequence was correct, and the Pet30a plasmid had been correctly inserted, resulting in a recombinant plasmid Pet30a-LysX (the corresponding recombinant expression vector). Plasmids with correct sequencing were stored in a refrigerator of −20° C. for later use.

If further used to produce a microorganism, the recombinant plasmid Pet30a-LysX described above can be introduced into Escherichia coli by genetic engineering to obtain the corresponding recombinant microorganism. The specific processing of the genetic engineering here is carried out in a conventional manner.

### Example 3

In order to determine the enzyme activity of the pyridoxal kinase mutant, the corresponding pyridoxal kinase mutant obtained by the method in Example 2 was selected, i.e., lysine at position 229 was mutated to the corresponding amino acid, resulting in the pyridoxal kinase mutants shown below, specifically K229A, K229C, K229D, K229E, K229F, K229H, K229I, K229L, K229M, K229N, K229P, K229R, K229S, K229T, K229V, K229G, K229W, and K229Y.

### Enzyme Activity Assay of Wild-Type PdxK and the Pyridoxal Kinase Mutant:

3 Ml of a reaction solution containing 20 g/L pyridoxal hydrochloride, 10 g/L an adenosine triphosphate disodium salt, 30 g/L an acetyl phosphate diammonium salt, and 2 g/L magnesium sulfate heptahydrate was taken and stirred to be dissolved, and then adjusted to Ph=5.0 by using a 10 M sodium hydroxide solution, 0.2 Ml of acetate kinase (a supernatant after disruption) and 0.2 Ml of a crude enzyme solution (a supernatant after disruption) were added, and the reaction solution was subjected to a reaction with shaking in a water bath shaker at 200 rpm at 35° C. After 15 min, a sample was taken and heated to be boiled for 5 min, the reaction was stopped, and the change in the concentration of pyridoxal phosphate in the reaction solution was determined by HPLC.

The crude enzyme solution corresponds to a crude enzyme solution of wild-type PdxK or a crude enzyme solution of the pyridoxal kinase mutant, respectively.

Enzyme activity is defined as the amount of an enzyme required to catalyze the production of 1 mol pyridoxal phosphate per minute under the reaction condition of 35° C.

### Comparison of Enzyme Activity of Wild-Type PdxK and Enzyme Activity of the Pyridoxal Kinase Mutant

The pyridoxal kinase mutant (PL kinase mutant) genetically engineered bacteria successfully constructed in Example 2 were cultured by the method in Example 1 to obtain thalli, and after disruption, a crude enzyme solution of the corresponding pyridoxal kinase mutant was obtained, data analysis was performed, and the enzyme activity data analysis results of pyridoxal kinase mutants are shown in Table 2.

TABLE 2

| Mutation site 229 | |
|---|---|
| (a corresponding | Enzyme |
| mutant) | activity U/L |
| K229A | 6.56 |
| K229C | 1.17 |
| K229D | 6.33 |
| K229E | 6.18 |
| K229F | 9.86 |
| K229H | 5.75 |
| K229I | 9.04 |
| K229L | 5.35 |
| K229M | 2.99 |
| K229N | 2.83 |
| K229P | 7.25 |
| K229R | 8.72 |
| K229S | 10.61 |
| K229T | 7.09 |
| K229V | 4.82 |
| K229W | 2.45 |
| K229Y | 9.75 |
| K229G | 0 |
| Wild type | 4.70 |

The results of the enzyme activity data for the mutants in Table 1 above show that by mutating the lysine at position 229, not all of the mutants have an enzyme activity superior to that of the wild type, and some mutants have an enzyme activity inferior to that of the wild type. The pyridoxal kinase mutants of the present disclosure can achieve improved enzyme activity performance, which is far superior to the enzyme activity level of the wild type, but the specific enzyme activity does not necessarily enable the conversion to pyridoxal phosphate to have a high concentration effect.

Meanwhile, the wild-type PdxK and K229F were specifically selected for analysis of enzyme protein expression, and compared. The specific analysis results are shown in FIG. 1. The results show that proteins of the mutant and the wild type are highly expressed and the protein solubility is better.

### Example 4

Crude enzyme solutions obtained after fermentation and ultrasonic disruption of wild-type PdxK genetically engineered bacteria and acetate kinase engineered bacteria (existing acetate kinase) were used as a catalyst for a reaction process for preparing pyridoxal phosphate, and result analysis was performed.

2.05 g of pyridoxal hydrochloride and 2.35 g of an acetyl phosphate diammonium salt were used as a substrate, 0.2 g of magnesium sulfate heptahydrate and 1.4 g of an adenosine triphosphate disodium salt were added, 100 Ml of water was added to dissolve the above materials, a reaction Ph was adjusted to 5.0 with 10 M NaOH, 2 g of a crude enzyme solution of PdxK and 2 g of a crude enzyme solution of acetate kinase were added, and a reaction was controlled to be carried out at a Ph of 5.0 with a 3 M NaOH solution at 35° C. 0.5 g of the acetyl phosphate diammonium salt (ACP) was supplementally added every 1 h during the reaction, and a sample was taken every half hour, and the concentration of a product was determined by HPLC after centrifugation. 22.18 g/L of pyridoxal phosphate was finally produced in 4 h through the reaction. Analysis of the concentration of reaction process products is shown in FIG. 2.

### Example 5

The pyridoxal kinase mutant was used as a catalyst for a reaction process for preparing pyridoxal phosphate, and result analysis was performed, this example was exemplified by a reaction using a K229F mutant.

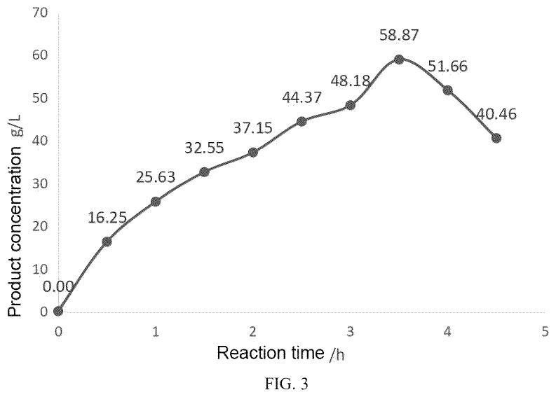

A total of 20 Ml of a reaction solution containing 1.00 g of pyridoxal hydrochloride, 0.28 g of an adenosine triphosphate disodium salt, 0.80 g of an acetyl phosphate diammonium salt, and 0.04 g of magnesium sulfate heptahydrate was charged into a 50 Ml round bottom reaction tube, 15.80 Ml of purified water was added, after stirring until dissolved, a Ph was adjusted to be 5.0 with 10 M sodium hydroxide, 0.50 g of acetate kinase and 1.00 g of a crude enzyme solution of a mutant K229F were added separately, and a reaction was carried out in a water bath shaker at 35° C., Ph=5.0, and 200 rpm. A sample was taken every half hour, the change in pyridoxal phosphate concentration was detected by HPLC, and 0.2 g of the acetyl phosphate diammonium salt was supplemented every half hour. The liquid phase detection results showed that the mutant K229F catalyzed the production of 58.87 g/L pyridoxal phosphate in 3.5 h. Analysis of the concentration of reaction process products is shown in FIG. 3.

### Example 6

### Summary Table of Reaction Results of Other Pyridoxal Kinase Mutants

Other pyridoxal kinase mutant engineered bacteria were subjected to fermentation in a 1 L shake flask and disruption to obtain crude enzyme solutions, and then the crude enzyme solution was used as a catalyst together with acetate kinase.

An enzyme-catalyzed reaction was carried out with pyridoxal hydrochloride as a substrate and an acetyl phosphate diammonium salt as a cosubstrate. A total of 20 Ml of a reaction solution having a Ph of 5.0 and containing 1.00 g of pyridoxal hydrochloride, 0.28 g of an adenosine triphosphate disodium salt, 0.80 g of an acetyl phosphate diammonium salt, and 0.04 g of magnesium sulfate heptahydrate was subjected to a reaction with shaking in a water bath at 35° C. and 200 rpm. The change in the concentration of a product pyridoxal phosphate was monitored by HPLC during the reaction process, and the concentrations of final products of the reaction can reach values shown in Table 3 below.

TABLE 3

| | | Product |
|---|---|---|
| Mutation | Reaction | concentration |
| site | time (h) | (g/L) |
| K229A | 8 | 43.92 |
| K229E | 8 | 24.24 |
| K229R | 8 | 30.66 |
| K229T | 7 | 36.39 |
| K229M | 7 | 33.54 |
| K229H | 3 | 41.53 |
| K229S | 0.4 | 13.82 |
| K229Y | 0.4 | 9.89 |
| K229I | 0.4 | 8.74 |
| K229N | 0.4 | 12.83 |
| K229P | 0.4 | 9.94 |
| K229C | 0.4 | 1.56 |
| K229W | 0.4 | 3.41 |
| K229V | 0.4 | 5.83 |
| K229D | 0.4 | 8.68 |
| K229L | 0.4 | 9.09 |

In conjunction with the tracking analysis of the corresponding reaction processes in Examples 4 and 5 above, and the level achieved by the concentration of the reaction product in this example, when the wild-type pyridoxal kinase and the pyridoxal kinase mutant of the present disclosure in FIGS. 2 and 3 are used to catalyze the phosphorylation of pyridoxal to synthesize pyridoxal phosphate, it can be clearly seen from the concentration of pyridoxal phosphate obtained during the catalytic conversion reaction of the corresponding wild type in FIG. 2 that the concentration of the product pyridoxal phosphate obtained through enzymatic catalysis by using the mutant of the present disclosure is much higher than the catalytic conversion capacity of the wild-type pyridoxal kinase, and some mutants can even increase the concentration of pyridoxal phosphate to a concentration level of 2 times or more. As can be seen from the data analysis in Table 3 above, only K229C, K229W and K229G in the mutants of the present disclosure have low catalytic efficiency, and the remaining mutants all show better application values.

The specific examples described in the present disclosure are merely illustrative of the spirit of the present disclosure. Those skilled in the art to which the present disclosure belongs may make various modifications or supplements to the specific examples described or adopt similar substitutions without departing from the spirit of the present disclosure or departing from the scope defined by the appended claims.

Although the present disclosure has been described in detail and specific examples have been cited, it will be apparent to those skilled in the art that various changes and modifications may be made without departing from the spirit and scope of the present disclosure.

What is claimed is:

1. A pyridoxal kinase mutant, wherein the pyridoxal kinase mutant has an amino acid sequence selected from the amino acid sequence as shown in SEQ ID NO: 1 in which lysine at position 229 is mutated to alanine, phenylalanine, arginine, threonine, histidine, serine, tyrosine, valine, leucine, isoleucine, proline, aspartic acid, or glutamic acid; and the pyridoxal kinase mutant is used to catalyze a synthesis of pyridoxal phosphate from pyridoxal.

2. The pyridoxal kinase mutant according to claim 1, wherein the pyridoxal kinase mutant is produced by mutating lysine at position 229 of an amino acid sequence derived from Escherichia coli having the amino acid sequence as shown in SEQ ID NO: 1 and the nucleotide sequence as shown in SEQ ID NO: 2.

3. A recombinant expression vector carrying the pyridoxal kinase mutant according to claim 1.

4. A microbial cell having a microorganism as an expression host, wherein the microorganism carries a pyridoxal kinase mutant or carries a recombinant expression vector containing the pyridoxal kinase mutant; the pyridoxal kinase mutant has an amino acid sequence selected from the amino acid sequence as shown in SEQ ID NO: 1 in which lysine at position 229 is mutated to alanine, phenylalanine, arginine, threonine, histidine, serine, tyrosine, valine, leucine, isoleucine, proline, aspartic acid, or glutamic acid; and the pyridoxal kinase mutant is used to catalyze a synthesis of pyridoxal phosphate from pyridoxal.

5. The microbial cell according to claim 4, wherein the microorganism is Escherichia coli.

6. The microbial cell according to claim 4, wherein the pyridoxal kinase mutant is produced by mutating lysine at position 229 of an amino acid sequence derived from Escherichia coli having the amino acid sequence as shown in SEQ ID NO: 1 and the nucleotide sequence as shown in SEQ ID NO: 2.

7. A use of a pyridoxal kinase mutant, a recombinant expression vector, and a microbial cell for preparing pyridoxal phosphate by phosphorylating pyridoxal hydrochloride under an action of pyridoxal kinase and acetate kinase, wherein the pyridoxal kinase mutant is selected from the pyridoxal kinase mutant according to claim 1; the recombinant expression vector carries the pyridoxal kinase mutant; and the microbial cell has a microorganism as an expression host, and the microorganism carries the pyridoxal kinase mutant or the recombinant expression vector.

8. The use of the pyridoxal kinase mutant, the recombinant expression vector, and the microbial cell according to claim 7, wherein a specific preparation method of the pyridoxal phosphate comprises:
carrying out a reaction in a reaction system of the pyridoxal hydrochloride, a magnesium salt, ATP, the pyridoxal kinase, the acetate kinase, and ACP to prepare the pyridoxal phosphate.

9. The recombinant expression vector according to claim 3, wherein the pyridoxal kinase mutant is produced by mutating lysine at position 229 of an amino acid sequence derived from Escherichia coli having the amino acid sequence as shown in SEQ ID NO: 1 and the nucleotide sequence as shown in SEQ ID NO: 2.

10. The microbial cell according to claim 5, wherein the pyridoxal kinase mutant is produced by mutating lysine at position 229 of an amino acid sequence derived from Escherichia coli having the amino acid sequence as shown in SEQ ID NO: 1 and the nucleotide sequence as shown in SEQ ID NO: 2.

11. The use of the pyridoxal kinase mutant, the recombinant expression vector, and the microbial cell according to claim 7, wherein the pyridoxal kinase mutant is produced by mutating lysine at position 229 of an amino acid sequence derived from Escherichia coli having the amino acid sequence as shown in SEQ ID NO: 1 and the nucleotide sequence as shown in SEQ ID NO: 2.

12. The use of the pyridoxal kinase mutant, the recombinant expression vector, and the microbial cell according to claim 7, wherein the microorganism is Escherichia coli.

13. The use of the pyridoxal kinase mutant, the recombinant expression vector, and the microbial cell according to claim 11, wherein a specific preparation method of the pyridoxal phosphate comprises:
carrying out a reaction in a reaction system of the pyridoxal hydrochloride, a magnesium salt, ATP, the pyridoxal kinase, the acetate kinase, and ACP to prepare the pyridoxal phosphate.

14. The use of the pyridoxal kinase mutant, the recombinant expression vector, and the microbial cell according to claim 12, wherein a specific preparation method of the pyridoxal phosphate comprises:
carrying out a reaction in a reaction system of the pyridoxal hydrochloride, a magnesium salt, ATP, the pyridoxal kinase, the acetate kinase, and ACP to prepare the pyridoxal phosphate.
